(19) United States
(12) Patent Application Publication
(10) Pub. No.: US 2025/0041021 A1
(43) Pub. Date: Feb. 6, 2025

(54) POSITIONAL MARKERS FOR CATHETER WIRES

(71) Applicant: Boston Scientific Medical Device Limited, Galway (IE)

(72) Inventors: Ahmad Kamal, Milton (CA); Robert Harrison, Milton (CA); Mahban Samiee-Zafarghandy, Etobicoke (CA); Yun Uhm, Toronto (CA); John Paul Urbanski, Toronto (CA); Saad Asif, Mississauga (CA); Christopher Bermingham, Etobicoke (CA)

(21) Appl. No.: 18/793,488

(22) Filed: Aug. 2, 2024

Related U.S. Application Data
(60) Provisional application No. 63/517,662, filed on Aug. 4, 2023.

(51) Int. Cl. A61B 90/00

(52) U.S. Cl. CPC A61B 90/39

(57) ABSTRACT

A transseptal access and crossing system for perforating a septum in a patient's heart includes an outer guide member having a distal end, a proximal hub, and a lumen extending therebetween. A radiofrequency perforation device includes a distal tip and a proximal portion. The radiofrequency perforation device is configured to translate within the lumen. The radiofrequency perforation device proximal portion includes a distal marker, an intermediate marker, and a proximal marker. Each of the distal marker, the intermediate marker, and the proximal marker are positioned and sized to provide an indication of the location of the distal tip of the radiofrequency perforation device in relationship to the distal end of the outer guide member.

### CROSS REFERENCE TO RELATED APPLICATIONS

This application claims priority to U.S. Provisional Patent Application No. 63/517,662 filed Aug. 4, 2023, which is incorporated herewith by reference in its entirety.

### TECHNICAL FIELD

The present invention relates generally to methods and devices usable within the body of a patient. More specifically, the present invention is concerned with a system and method for providing positional cues for the positioning of medical devices.

### BACKGROUND

Fluoroscopic imaging is commonly used during medical procedures. However, radiation from fluoroscopic imaging can be harmful. As such, there is a need to reduce the amount of radiation from fluoroscopic imaging and enable fluoro-less cardiac procedures. Markers positioned along catheter guidewires can be used as positional cues as an alternative to using fluoroscopic imaging so that a user may ascertain a location or configuration of the guidewires during use.

A simple solid marker on the guidewire provides limited information. With physicians looking for different cues at different stages of a procedure, the meaning of the marker can be perceived incorrectly or be confusing to the user. There is a need for a system to better enable fluoro-less cardiac procedures.

### SUMMARY

Example 1 is a transseptal access and crossing system for perforating a septum in a patient's heart including an outer guide member having a distal end, a proximal hub, and a lumen extending therebetween. A radiofrequency perforation device includes a distal tip and a proximal portion. The radiofrequency perforation device is configured to translate within the lumen. The radiofrequency perforation device proximal portion includes a distal marker, an intermediate marker, and a proximal marker. Each of the distal marker, the intermediate marker, and the proximal marker are positioned and sized to provide an indication of the location of the distal tip of the radiofrequency perforation device in relationship to the distal end of the outer guide member.

Example 2 is the system of Example 1 wherein the distal marker, the intermediate marker, and the proximal marker are asymmetrically positioned along the proximal portion.

Example 3 is the system of Example 1 wherein the distal marker, the intermediate marker, and the proximal marker are symmetrically positioned along the proximal portion.

Example 4 is the system of any of Examples 1-3 wherein a distal edge of the distal marker is configured to provide an indication that the distal tip of the radiofrequency perforation device is located within the outer guide member.

Example 5 is the system of any of Examples 1-4 wherein a center of the intermediate marker is configured for gripping.

Example 6 is the system of any of Examples 1-5 further including a distal intermediate marker and a proximal intermediate marker, the distal intermediate marker and the proximal intermediate marker being configured to provide an indication of the distal tip of the radiofrequency perforation device being advanced out of the outer guide member.

Example 7 is the system of any of Examples 1-6 wherein the wherein the outer guide member comprises a catheter, dilator, or a sheath.

Example 8 is the system of any of Examples 1-7 wherein the outer guide member includes a surface configured to align with a portion of the distal marker, the proximal marker, and the intermediate marker.

Example 9 is the system of any of Examples 1-8 further including one or more tactile markers located on the radiofrequency perforation device proximal portion.

Example 10 is the system of Example 9 wherein the one or more tactile markers includes one or more recessed or raised features.

Example 11 is the system of any of Examples 1-10 wherein the distal marker and the intermediate marker have a same length.

Example 12 is the system of any of Examples 1-10 wherein the distal marker and the intermediate marker have different lengths.

Example 13 is the system of any of Examples 1-12 wherein a length of the distal marker, the intermediate marker, or the proximal marker is in the range of 1 mm to 8 mm.

Example 14 is the system of any of Examples 1-12 wherein a length of the distal marker, the intermediate marker, or the proximal marker is in the range of 4 mm to 12 mm.

Example 15 is the system of any of Examples 1-14 wherein the radiofrequency perforation device does not include a handle coupled to the proximal portion.

Example 16 is a transseptal access and crossing system for perforating a septum in a patient's heart. The system includes an outer guide member having a distal end and a proximal hub, and a lumen extending therebetween. A radiofrequency perforation device includes a distal tip and a proximal portion. The radiofrequency perforation device is configured to translate within the lumen. The radiofrequency perforation device proximal portion includes a distal marker, an intermediate marker, and a proximal marker. Each of the distal marker, the intermediate marker, and the proximal marker are positioned and sized to provide an indication of the location of the distal tip of the radiofrequency perforation device in relationship to the distal end of the outer guide member. The distal marker, the intermediate marker, and the proximal marker include contrasting colors.

Example 17 is the system of Example 16 wherein the distal marker, the intermediate marker, and the proximal marker are asymmetrically positioned along the proximal portion

Example 18 is the system of Example 16 wherein the distal marker, the intermediate marker, and the proximal marker are symmetrically positioned along the proximal portion.

Example 19 is the system of Example 16 wherein a distal edge of the distal marker is configured to provide an indication that the distal tip of the radiofrequency perforation device is located within the outer guide member.

Example 20 is the system of Example 16 wherein a center of the intermediate marker is configured for gripping.

Example 21 is the system of Example 16 further including a distal intermediate marker and a proximal intermediate marker, the distal intermediate marker and the proximal intermediate marker being configured to provide an indication of the distal tip of the radiofrequency perforation device being advanced out of the outer guide member.

Example 22 is the system of Example 16 wherein the outer guide member comprises a catheter, dilator, or a sheath.

Example 23 is the system of Example 16 wherein the outer guide member includes a surface configured to align with a portion of the distal marker, the proximal marker, and the intermediate marker.

Example 24 is the system of Example 16 further including one or more tactile markers located on the radiofrequency perforation device proximal portion.

Example 25 is the system of Example 24 wherein the one or more tactile markers includes one or more recessed or raised features.

Example 26 is the system of Example 16 wherein the distal marker and the intermediate marker have a same length.

Example 27 is the system of Example 16 wherein the distal marker and the intermediate marker have different lengths.

Example 28 is the system of Example 16 wherein a length of the distal marker, the intermediate marker, or the proximal marker is in the range of 1 mm to 8 mm.

Example 29 is the system of Example 16 wherein a length of the distal marker, the intermediate marker, or the proximal marker is in the range of 4 mm to 12 mm.

Example 30 is the system of Example 16 wherein the radiofrequency perforation device does not include a handle coupled to the proximal portion.

Example 31 is a transseptal access and crossing system for perforating a septum in a patient's heart that includes an elongate member having a distal tip and a proximal portion. The proximal portion includes a distal marker, an intermediate marker, and a proximal marker. The distal marker, the intermediate marker, and the proximal marker are configured to provide an indication of the location of the inner member in relationship to an outer member. The distal marker, the intermediate marker, and the proximal marker include contrasting colors.

Example 32 is the system of Example 31 wherein the distal marker, the intermediate marker, and the proximal marker are asymmetrically positioned along the proximal portion.

Example 33 is the system of Example 31 wherein the distal marker, the intermediate marker, and the proximal marker are symmetrically positioned along the proximal portion.

Example 34 is a transseptal access and crossing system for perforating a septum in a patient's heart including an elongate member having a distal tip and a proximal portion. The proximal portion includes a distal marker, a distal intermediate marker, an intermediate marker, a proximal intermediate marker, and a proximal marker. The distal marker, the distal intermediate marker, the intermediate marker, the proximal intermediate marker, and the proximal marker are configured to provide an indication of the location of the inner member in relationship to an outer member.

Example 35 is the system of Example 34 wherein the distal marker, the distal intermediate marker, the intermediate marker, the proximal intermediate marker, and the proximal marker are symmetrically positioned along the proximal portion.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2A illustrates an inner member being positioned within an outer member.

FIG. 2B illustrates an inner member extending from an outer member such that a distal tip of the inner member is exposed.

FIG. 3 illustrates a system 300 for providing positional cues for the location of an inner member in relationship to an outer member in accordance with an embodiment.

FIG. 4 illustrates a system 400 for providing positional cues for the location of an inner member in relationship to an outer member in accordance with an embodiment.

FIG. 5 illustrates a system 500 for providing positional cues for the location of an inner member in relationship to an outer member in accordance with an embodiment.

FIG. 6 illustrates a system 600 for providing positional cues for the location of an inner member in relationship to an outer member in accordance with an embodiment.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

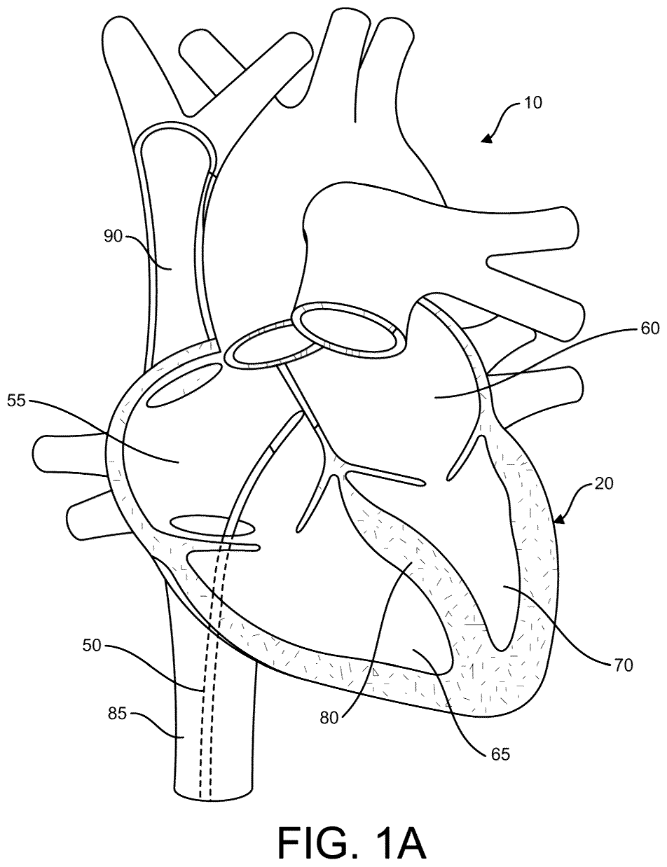

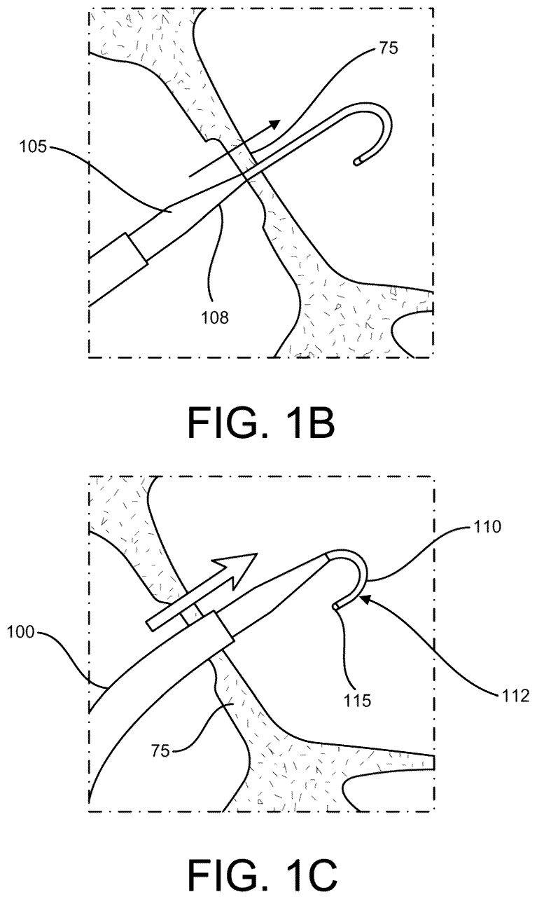

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a piercing device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In another embodiment, the RF perforation device 110 is inserted in the SVC 90. Next, the sheath 100 and dilator 105 are inserted over the RF perforation device 110 and the RF perforation device 110 is withdrawn inside the dilator 105. In this configuration the assembly is withdrawn from the SVC 90 and positioned at a desired location against the to the atrial septum 75. The RF perforation device 110 can be advanced out of the dilator 105 to puncture the septum 75.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the RF generator is electrically coupled to the RF perforation device 110 using a clip that is removable coupled to a proximal portion of the performation device 110. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In the various embodiments disclosed herein, the perforation device 110 does not include a hub or handle connected to a proximal portion of the wire, which could function as a positional indicator for the perforation device, for example, by allowing a user to position such a hub or handle with respect to a corresponding structure on the outer member. Thus, to allow a user to properly position an inner member, such as a guidewire or RF perforation device 110, in relationship to the dilator 105, a system of markers may be placed on a proximal portion of the inner member to provide cues to a user. The system of markers may include a plurality of markers positioned symmetrically or asymmetrically along the proximal portion of the inner member. The markers may include a plurality of different colors or different tactile cues The plurality of different colors may include a first color and a second color that are contrasting and easily discernable from one another, for example white and blue.

The markers may provide cues to a user in order to allow for proper positioning of an inner member within an outer member during different steps of a medical procedure. For example, multiple markers can be used to indicate positioning of an inner member such that a distal tip is contained within the outer member, indicate proper positioning of a distal tip during RF delivery, indicate proper positioning of a distal tip for tenting tissue, indicate a recommended insertion depth into a pulmonary vein, indicate an insertion depth until max rail of the inner member, or indicate when the tip of the inner member is protruding a set distance from the outer member.

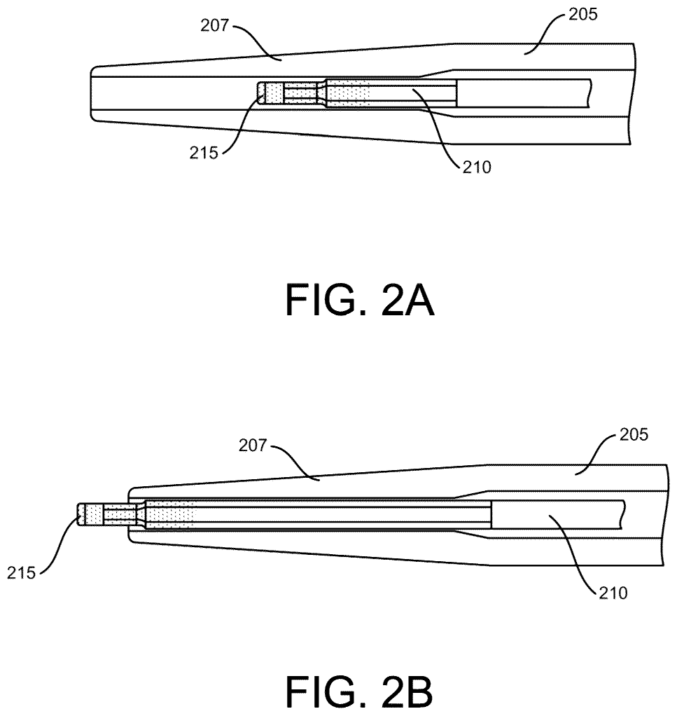

FIG. 2A illustrates an inner member 210, such as a guidewire or RF perforation device, for example, positioned within an outer member 205 (e.g., a guide member), such as a sheath, catheter, or dilator, during a portion of a medical procedure. The outer member 205 may include a tapered distal portion 207. As illustrated in FIG. 2A, the inner member 210 is located within the outer member 205 such that the distal tip 215 of the inner member 210 is located within the outer member 205. In this configuration, the outer member 205 and the inner member 210 may be moved throughout a patient's vasculature to a desired location. In some aspects, the desired location may be within a right atrium adjacent a patients atrial septum 75.

FIG. 2B illustrates the inner member 210 extending from the outer member 205 such that the distal tip 215 of the inner member 210 is exposed. In some aspects, the inner member 210 may include an RF perforation or puncture device and the distal tip 215 may include an RF electrode configured to puncture tissue. The configuration shown in FIG. 2B may be useful when performing a transseptal crossing procedure, allowing the distal tip 215 to puncture a patient's atrial septum 75. In some embodiments, the inner member 210 is a mechanical puncturing device, such as a needle having a sharp tip that is configured to pierce or puncture tissue.

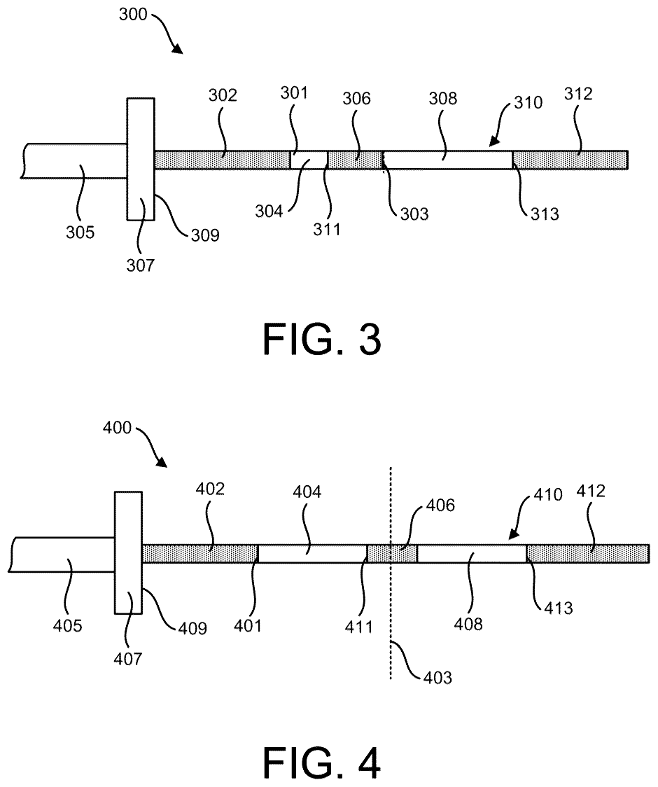

FIG. 3 illustrates a system 300 for providing positional cues for the location of an inner member 310 in relationship to an outer member 305 in accordance with an embodiment. System 300 includes asymmetric markers located on a proximal portion of the inner member 310 to aid a user in positioning the inner member 310 within the outer member 305. The markers are situated asymmetrically towards a proximal end of the inner member 310. As illustrated in FIG. 3, the proximal portion of the inner member 310 includes a distal portion 302, a proximal portion 312, and a plurality of markers 304, 306, 308 located therebetween. According to various embodiments, the plurality of markers includes a distal marker 304, an intermediate marker 306, and a proximal marker 308.

Outer member 305 includes a lumen into which the inner member 310 translates. A hub 307 at the proximal end of the outer member 305 provides a handle or gripping portion that a user can use to manipulate the outer member 305 with respect to the inner member 310. The hub 307 includes a proximal surface 309 that acts as a reference for the plurality of markers 304, 306, 308.

In FIG. 3, the inner member 310 may include a coating having a first color. In this configuration, the distal wire portion 302, intermediate marker 306, and proximal wire portion 312 may include the first color. Distal marker 304 and proximal marker 308 may be formed by placing a thin layer of a material having a second color, which contrasts the first color, over the inner member 310. In this manner, visual cues are created to allow a user to properly advance or withdrawal the inner member 310 out of or into the outer member during a procedure. In some aspects, the first color may comprise blue or another dark color and the second color may include white or another light color. In some aspects, the intermediate marker 306 may include a third color that is different from the first and second color.

Alternatively, a method of forming the markers may include coating the inner member 310 in a material having a first color, such as white or another light color. Next, portions of the inner member 310 defining markers 304 and 308 may be masked. After which a material having a second color, such as blue or another dark color, may be applied to the inner member 310. Once the masking material is removed, markers 304 and 308 will provide contrasting visual cues to a user.

In some aspects, the distal marker 304 and the intermediate marker 306 may have a same length. In other aspects, distal marker 304 and intermediate marker 306 may have different lengths. In some aspects, the length of the distal marker 304 and the intermediate marker 306 may be in the range of approximately 1 mm to 8 mm and the length of the proximal marker 308 may be in the range of approximately 4 mm to 10 mm. For example, the distal marker 304 can be a 3 mm white marker, followed by the intermediate marker 306 being a 3 mm blue marker, followed by the proximal marker 308 being a 6 mm white marker. In another example, the distal marker 304 can be a 4 mm white marker, followed by a blue 4 mm intermediate marker 306, followed by a white 4 mm proximal marker 308. In some aspects, the distal marker 304 and the intermediate marker 306 may have lengths greater than 10 mm.

The system 300 is configured such that a distal most edge 301 of the distal marker 304 indicates when the distal tip of the inner member 310 is located within the outer member 305. As such, when the distal most edge 301 is aligned with the proximal surface 309 of the hub 307, a user knows that the distal tip of the inner member 310 is safely stored within the outer member 305. Additionally, a distal most edge 303 of the proximal marker 308 indicates when the distal tip of the inner member 310 is advanced from the outer member 305 a desired length, for example for tenting tissue in preparation for the delivery of RF energy or to indicate that the distal tip is extended a desired amount appropriate for puncturing tissue, such as 1-3 mm. As such, when the distal most edge 303 of the proximal marker is aligned with the proximal surface 309 of the hub 307, a user knows that the distal tip of the inner member 310 is protruding from the outer member 305 a desired distance.

In some aspects, a proximal most edge 311 of the distal marker 304 may provide an indication of the location of the inner member 310 in relationship to the outer member 305. For example, when the proximal most edge 311 of the distal marker 304 is aligned with the proximal surface 309 of the hub 307, a user can expect the distal tip of the inner member 310 to be flush with the outer member 305. Additionally, a proximal most edge 313 of the proximal marker 308 may provide an indication of the location of the inner member 310 in relationship to the outer member 305. For example, when the proximal most edge 313 of the proximal marker 308 is aligned with the proximal surface 309 of the hub 307, a user can expect the distal tip of the inner member 310 to be protruding from the outer member 305 such that a pre-formed section of the inner member achieves a pre-formed shape.

FIG. 4 illustrates a system 400 for providing positional cues for the location of an inner member 410, such as a guidewire, RF perforation device, or mechanical puncturing device in relationship to an outer member 405, such as a catheter, sheath, or dilator, in accordance with an embodiment. System 400 includes symmetric markers to aid a user in positioning the inner member 410 within the outer member 405 during a procedure. As illustrated in FIG. 4, the proximal portion of the inner member 410 includes a distal wire portion 402, a proximal wire portion 412, and a plurality of markers 404, 406, 408 located therebetween. The plurality of markers includes a distal marker 404, an intermediate marker 406, and a proximal marker 408. In some embodiments, the distal marker 404 and the proximal marker 408 are contrasting colors or materials from the wire which forms the distal wire portion 402, the intermediate marker 406, and the proximal wire portion. In some aspects, the distal marker 404, the intermediate marker 406, and the proximal marker 408 are formed of two or more contrasting colors.

The outer member 405 includes a lumen into which the inner member 410 translates. A hub 407 at the proximal end of the outer member 405 provides a handle or gripping portion that a user can grab to manipulate the outer member 405 with respect to the inner member 410. The hub 407 includes a proximal surface 409 that acts as a reference for the plurality of markers 404, 406, 408.

As illustrated in FIG. 4, the distal marker 404 and the intermediate marker 406 have a same length. In some aspects, the length of the distal marker 404 and the proximal marker 408 may be in the range of approximately 4 mm to 12 mm. Intermediate marker 406 has a length that is shorter than the length of the distal marker 404 and the proximal marker 408.

The system 400 is configured such that a distal most edge 401 of the distal marker 404 indicates when the distal tip of the inner member 410 is located within the outer member 405. As such, when the distal most edge 401 is aligned with the proximal surface 409 of the hub 407, a user is aware that the distal tip of the inner member 410 is safely stored within the outer member 405. Other edges of the distal marker 404, the intermediate marker 406, and the proximal marker 408 may also be used to indicate positioning of the inner member 410 within the outer member 405 as discussed above. Likewise, a user may choose to use the center of each marker as a reference.

Additionally, system 400 is configured to indicate where the user is to hold the inner member 410 during a particular part of a procedure. For example, the intermediate marker 406 can be gripped in the center 403 during RF energy delivery. By doing so, the user will be unable to advance the inner member 410 past a certain distance because the user's fingers will contact the proximal surface 409 of the hub 407. Normally, if the RF delivery lasts too long or if the user advances an RF wire too fast, the inner member 410 can curl back onto the tissue it punctures through and create unnecessary defects. By griping the inner member at the center 403 of the intermediate marker 406 and advancing until contacting the proximal surface, one can eliminate the likelihood of the inner member 410 curling back.

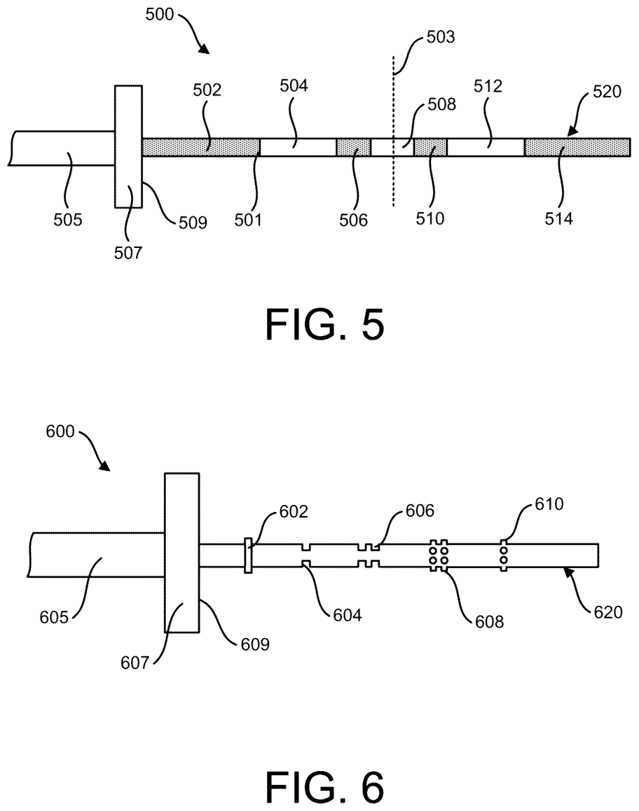

FIG. 5 illustrates a system 500 for providing positional cues for the location of an inner member 520, such as a guidewire or RF perforation device, in relationship to an outer member 505, such as a catheter, sheath, or dilator, in accordance with an embodiment. System 500 includes symmetric markers to aid a user in positioning the inner member 520 within the outer member 505 during a procedure. As illustrated in FIG. 5, the proximal portion of the inner member 520 includes a distal wire portion 502, a proximal wire portion 514, and a plurality of markers 504, 506, 508, 510, 512 located therebetween. The plurality of markers includes a distal marker 504, a distal intermediate marker 506, an intermediate marker 508, a proximal intermediate marker 510, and a proximal marker 512.

Outer member 505 includes a lumen into which the inner member 520 translates. A hub 507 at the proximal end of the outer member 505 provides a handle or gripping portion that a user can grab to manipulate the outer member 505 with respect to the inner member 520. The hub 507 includes a proximal surface 509 that acts as a reference for the plurality of markers 504, 506, 508, 510, 512.

As illustrated in FIG. 5, the distal marker 504 and the intermediate marker 512 have a same length. In some aspects, the length of the distal marker 504 and the proximal marker 512 may be in the range of approximately 2 mm to 10 mm. The distal intermediate marker 506, the intermediate marker 508, and the proximal intermediate marker 510 have a length that is shorter than the length of the distal marker 504 and the proximal marker 508. In some aspects, the distal intermediate marker 506, the intermediate marker 508, and the proximal intermediate marker 510 have a same length. In some aspects, the distal intermediate marker 506 and the proximal intermediate marker 510 have a same length that is different from a length of the intermediate marker 508.

The system 500 is configured such that a distal most edge 501 of the distal marker 504 indicates when the distal tip of the inner member 520 is located within the outer member 505. As such, when the distal most edge 501 is aligned with the proximal surface 509 of the hub 507, a user is aware that the distal tip of the inner member 520 is safely located within the outer member 505. As discussed above, edges of the distal marker 504, the distal intermediate marker 506, the intermediate marker 508, the proximal intermediate marker 510, and the proximal marker 512 may be used to indicate different lengths of protrusion of the inner member 520 from the outer member 505.

Additionally, system 500 is configured to indicate where the user is to hold the inner member 520 during a particular part of a procedure. For example, the intermediate marker 508 can be gripped in the center 503 during RF energy delivery. By doing so, the user will be unable to advance the inner member 520 past a certain distance because the user's fingers will contact the proximal surface 509 of the hub 507. Thus, eliminating the likelihood of the inner member 520 curling back onto tissue.

In some aspects, tactile markers may be added to the proximal portion of an inner member to provide a user with a tactile cue. In some aspects the tactile markers may be in addition to the visual markers of the system, while in other aspects the visual markers may be replaced by tactile markers.

In some aspects, the tactile markers may include one or more raised portion, such as a bump or ridge. In other aspects, the tactile markers may include one or more recessed portion, such as an opening or channel.

FIG. 6 illustrates a system 600 for providing positional cues for the location of an inner member 620, such as a guidewire or RF perforation device, in relationship to an outer member 605, such as a catheter, sheath, or dilator in accordance with an embodiment. The proximal portion of the inner member 620 includes a plurality of tactile markers 602, 604, 606, 608, 610. Each of the plurality of tactile markers 602, 604, 606, 608, 610 is configured to provide an indication of a location of the inner member 620 in relationship to the outer member 605.

The outer member 605 includes a lumen into which the inner member 620 translates. A hub 607 at the proximal end of the outer member 605 provides a handle or gripping portion that a user can grab to manipulate the outer member 605 with respect to the inner member 620. The hub 607 includes a proximal surface 609 that acts as a reference for the plurality of tactile markers 602, 604, 606, 608, 610. A user may grasp one of the plurality of tactile markers 602, 604, 606, 608, 610 and advance the inner member 620 until the user's fingers contact the proximal surface 609.

The plurality of tactile markers 602, 604, 606, 608, 610 includes a first tactile marker 602, a second tactile marker 604, a third tactile marker 606, a fourth tactile marker 608, and a fifth tactile marker 610. Each marker is configured to provide an indication of the location of the inner member 620 with respect to the outer member 605. The first tactile marker 602 includes a single raised ring feature and is configured to indicate when the distal tip of the inner member 620 is located within the outer member 605. The second tactile marker 604 includes a single recessed channel that extends around the inner member 620. The second tactile marker 604 is configured to indicate when the distal tip of the inner member 620 is positioned flush with the distal tip of the outer member 605. The third tactile marker 606 includes a plurality of recessed channels extending around the inner member 620. The fourth tactile marker 608 includes a plurality of raised features arranged in two rows around the inner member 620. Both the third tactile marker 606 and the fourth tactile marker 608 are configured to indicate that the distal tip of the inner member 620 is extending from the outer member 605 at desired amounts. The fifth tactile marker 610 includes a plurality of raised features, such as bumps, arranged in a single row around the inner member 620. The fifth tactile marker 610 is configured to indicate that the inner member 620 is extended such that a pre-formed curve of the inner member 620 is obtained. It is understood that while each of the five tactile makings are configured differently, that each one could be formed the same.

In some aspects, the inner or outer members may include one or more markers along a distal portion thereof for identifying a position or location during use using an imaging modality.

In some aspects, the inner or outer members may include a plurality of cuts machined into a wall thereof, for example by laser cutting. The shape and positioning of the cuts can allow for a transition in flexibility from a proximal portion to distal portion. The cuts may include a broken spiral configuration or may be positioned substantially orthogonal to a longitudinal axis of the inner or outer members. In some aspects, there may be a single cut that winds around an axis with a wider spacing between loops at the proximal portion and a larger spacing at the distal portion. The spacing and size of the cuts can be varied to achieve different flexibilities along the length of the inner or outer members.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A transseptal access and crossing system for perforating a septum in a patient's heart, the system comprising:
an outer guide member having a distal end and a proximal hub, and a lumen extending therebetween;
a radiofrequency perforation device having a distal tip and a proximal portion, the radiofrequency perforation device being configured to translate within the lumen;
the radiofrequency perforation device proximal portion comprising a distal marker, an intermediate marker, and a proximal marker;
wherein each of the distal marker, the intermediate marker, and the proximal marker are positioned and sized to provide an indication of the location of the distal tip of the radiofrequency perforation device in relationship to the distal end of the outer guide member, and
wherein the distal marker, the intermediate marker, and the proximal marker include contrasting colors.

2. The system of claim 1, wherein the distal marker, the intermediate marker, and the proximal marker are asymmetrically positioned along the proximal portion.

3. The system of claim 1, wherein the distal marker, the intermediate marker, and the proximal marker are symmetrically positioned along the proximal portion.

4. The system of claim 1, wherein a distal edge of the distal marker is configured to provide an indication that the distal tip of the radiofrequency perforation device is located within the outer guide member.

5. The system of claim 1, wherein a center of the intermediate marker is configured for gripping.

6. The system of claim 1, further comprising:
a distal intermediate marker and a proximal intermediate marker, the distal intermediate marker and the proximal intermediate marker being configured to provide an indication of the distal tip of the radiofrequency perforation device being advanced out of the outer guide member.

7. The system of claim 1, wherein the outer guide member comprises a catheter, dilator, or a sheath.

8. The system of claim 1, wherein the outer guide member includes a surface configured to align with a portion of the distal marker, the proximal marker, and the intermediate marker.

9. The system of claim 1, further comprising:
one or more tactile markers located on the radiofrequency perforation device proximal portion.

10. The system of claim 9, wherein the one or more tactile markers includes one or more recessed or raised features.

11. The system of claim 1, wherein the distal marker and the intermediate marker have a same length.

12. The system of claim 1, wherein the distal marker and the intermediate marker have different lengths.

13. The system of claim 1, wherein a length of the distal marker, the intermediate marker, or the proximal marker is in the range of 1 mm to 8 mm.

14. The system of claim 1, wherein a length of the distal marker, the intermediate marker, or the proximal marker is in the range of 4 mm to 12 mm.

15. The system of claim 1, wherein the radiofrequency perforation device does not include a handle coupled to the proximal portion.

16. A transseptal access and crossing system for perforating a septum in a patient's heart, the system comprising:
an elongate member having a distal tip and a proximal portion;
the proximal portion comprising a distal marker, an intermediate marker, and a proximal marker;
wherein the distal marker, the intermediate marker, and the proximal marker are configured to provide an indication of the location of the inner member in relationship to an outer member, and
wherein the distal marker, the intermediate marker, and the proximal marker include contrasting colors.

17. The system of claim 16, wherein the distal marker, the intermediate marker, and the proximal marker are asymmetrically positioned along the proximal portion.

18. The system of claim 16, wherein the distal marker, the intermediate marker, and the proximal marker are symmetrically positioned along the proximal portion.

19. A transseptal access and crossing system for perforating a septum in a patient's heart, the system comprising:
an elongate member having a distal tip and a proximal portion;
the proximal portion comprising a distal marker, a distal intermediate marker, an intermediate marker, a proximal intermediate marker, and a proximal marker;
wherein the distal marker, the distal intermediate marker, the intermediate marker, the proximal intermediate marker, and the proximal marker are configured to provide an indication of the location of the inner member in relationship to an outer member.

20. The system of claim 19, wherein the distal marker, the distal intermediate marker, the intermediate marker, the proximal intermediate marker, and the proximal marker are symmetrically positioned along the proximal portion.
